(19) United States
(12) Patent Application Publication
(10) Pub. No.: US 2013/0164337 A1
(43) Pub. Date: Jun. 27, 2013

(54) ANTIMICROBIAL ACTION PHARMACEUTICAL COMPOSITION FOR PARENTERAL ADMINISTRATION AND ITS PRODUCTION PROCESS

(71) Applicants: Viktor Lvovich Limonov, Moscow (RU); Konstantin Valentinoyich Gaidul, Novosibirsk (RU); Aleksandr Valerevich Dushkin, Novosibirsk (RU)

(72) Inventors: Viktor Lvovich Limonov, Moscow (RU); Konstantin Valentinoyich Gaidul, Novosibirsk (RU); Aleksandr Valerevich Dushkin, Novosibirsk (RU)

(21) Appl. No.: 13/390,151

(22) PCT Filed: May 11, 2011
(86) PCT No.: PCT/RU11/00320

§ 371 Date: Feb. 13, 2012

(30) Foreign Application Priority Data
Sep. 13, 2010 (RU) 201001449

(51) Int. Cl. A61K 9/14; A61K 31/427; A61K 31/407; A61K 31/546; A61K 31/43

(52) U.S. Cl. CPC A61K 9/145; A61K 31/546; A61K 31/43; A61K 31/407; A61K 31/427
USPC 424/400; 514/206; 514/202; 514/197; 514/203; 514/210.13; 514/210.15

(57) ABSTRACT

There is proposed herein a process for production of composite antimicrobial preparations for parenteral administration, featuring a higher therapeutic efficiency in case of grave infection and inflammatory diseases. The proposed compositions include active agents being betalactam antibiotics and finely dispersed nanostructured silica dioxide with a weight ratio from 10:1 to 75:1 respectively. The silica dioxide particles are antibiotic molecules delivery agents into the phagocytes, which allows increasing the antimicrobial preparations concentration at inflammation areas and considerably decrease microorganisms antibiotic resistance. The mentioned production process includes mixing betalactam antibiotic with finely dispersed nanostructured silica dioxide. Its main difference is that the mentioned substances mixture with a weight ratio from 10:1 to 75:1 respectively is exposed to mechanical processing by impact and abrasive actions until a proportion of the fine powder fraction with particles smaller than 5 micrometers increases to at least 25%. The so obtained mixture is used for injection preparations.

### CROSS-REFERENCE TO RELATED APPLICATIONS

This application is a U.S. national stage application of a PCT application PCT/RU2011/000320 filed on 11 May 2011, whose disclosure is incorporated herein in its entirety by reference, which PCT application claims priority of a EAPO application EA201001449 filed on 13 Sep. 2010.

### FIELD OF THE INVENTION

This invention belongs to antimicrobial pharmaceutical preparations and its' production technologies. It can be used in medicine and veterinary science for treating contagious and inflammatory diseases, as well as being used in pharmaceutical industry for medicinal products manufacturing.

### BACKGROUND OF THE INVENTION

Currently most contagious and inflammatory diseases successful therapy is based on using different anti-infectives, including beta-lactam antibiotics.

Beta-lactam are preparations (natural and semisynthetic penicillins, cephalosporins, cephamycins, carbapenems and monobactams) with a beta-lactam ring as a chemical structure common fragment, which determines the antimicrobial activity and a series of common properties of this drug preparation group [Used Literature 1].

All beta-lactam possess a wide antimicrobial spectrum and a high level of antimicrobial activity, but many of them have a fast developing microbial resistance, because of their specific ferments production—beta-lactamase (extended spectrum beta-lactamase, chromosomal beta-lactamase class C, etc.), which hydrolyze the beta-lactam ring. This is what deprives these preparations of their antibacterial properties and leads to microbe resistant strains development [2].

In the past decades there have been created specific beta-lactamase inhibitors (clavulanic acid, sulbactam, tazobactam and etc.) and on their basis there has been developed an entire range of effective combined antibacterial beta-lactam preparations of penicillin and cephalosporin family (amoxicillin/clavulanic acid, ampicillin/sulbactam, piperacillin/tazobactam, cefoperazone/sulbactam and etc.) which are noted because of their increased persistence to beta-lactamase as well as their more apparent antibacterial activity [2, 3].

Nevertheless it ought to be remarked that many of these “inhibitor screened” preparations appeared to be insufficiently effective because in case of high beta-lactamase production by germs the inhibitors cannot fully protect the antibiotics from hydrolysis.

The carbapenems which are resistant to many beta-lactamase action cannot entirely solve the microbial resistance to the mentioned antibiotics problem. It happens because many application ways for treating serious infections lead to forming of multiply P. Aeruginosa resistant strains [3].

Besides, frequently the clinical betalactam ineffectiveness (or their low effectiveness) in case of infections induced by different microbes is associated not only with the negative beta-lactamase activity, but also with these preparations limited ability of local concentration at contagious inflammation locus and macrophage penetration, where many contagious and inflammatory diseases' activators are deposited. The antimicrobial resistance level depends on their functional status intensity [4, 5].

In the last few years it has been discovered that the use of different nanoparticles as dosing vehicle for different antibiotics delivery (as well as betalactam) inside the bacteria and macrophages to increase their concentration at the contagious inflammation area and to increase their antimicrobial properties as well as phagocytes (neutrophils and macrophages) functional activity stimulation and their additional recruitment to infected tissues, is a very challenging trend for modern experimental pharmacology and clinical medicine [6, 7, 8, 9, 10, 11, 12].

### DESCRIPTION OF THE INVENTION

Here is the character of the mentioned invention. To increase the betalactam therapeutic effectiveness it is suggested to use the SiO₂ (silica dioxide) nanoparticles, which have different pharmacologically beneficial biocompatibility, biodistribution, biodegradation and low toxicity properties (independent from looseness of the structure intensity), can serve as antibiotics carrier for endocellular macrophage delivery, which are concentrated at the inflammatory tissues of lungs, liver, kidneys, lien, absorbent glands, heart, skin, bladder and other mammal organs (i.e. considerably increase antibiotics concentration in the infected areas), and also initiate the immune system cells antimicrobial activity. This will help to authentically increase the germicides therapeutic effect during contagious inflammatory diseases treatment [13, 14, 15, 16, 17, 18, 19, 20, 21].

The mentioned invention solves the issue of creating an antimicrobial action pharmaceutical composition for injections on basis of using betalactam and silica dioxide nanoparticles antibiotics which possess a higher therapeutic effectiveness (comparing to the standard betalactam which are considered as a basis for this invention) for contagious and inflammatory diseases treatment.

To solve the assigned task it is suggested to use an antimicrobial action pharmaceutical composition for injections, which contains a betalactam antibiotic and finely dispersed nanostructured silica dioxide w/w (10-75): 1.

The production process suggested to solve the assigned task is to obtain the antimicrobial action pharmaceutical composition for injections by mixing the betalactam antibiotics with other components. The betalactam antibiotic powder is mixed with the finely dispersed nanostructured silica dioxide powder w/w (10-75): 1. The procured mixture is machined by impact abrasive method.

The therapeutic effectiveness of the proposed pharmaceutical composition will increase if the obtained mixture is machined by abrasive method in a way that the part of finely dispersed nanostructured silica dioxide particles of 5 microns would be no less than 25%.

To prepare the mentioned pharmaceutical composition, were used foreign production antibiotics provided by Russian pharmacological company LLC “ABOLmed” (penicillins: carbenicillin; cephalosporins: cefazolin, cefuroxime, cefotaxime, ceftriaxone, cefoperazone, ceftazidime, cefoperazone/sulbactam, cefepime; cephamycims: cefoxin; carbapenems: meropenem; monobactams: aztreonam). As a finely dispersed nanostructured silica dioxide (hereafter referred to as BHSiO₂) was used “Polysorb” drug (pharmacological group: enterosorbing solution; active substance: colloidal silica dioxide), produced by Russian company CJSC “Polysorb”, containing round shaped silica dioxide nanoparticles (dimension 5-20 nm) combined into aggregates (irregular microparticles) with dimension ≦90 micron (registration number [symbol] 001140/01-100908). There is s similar preparation produced by Ukrainian company CJSC “Biopharma” with a trade name “Silics” [12].

The composition formulation choice was based on convertible betalactam molecules and nano- as well as micro BHSiO₂ particles sorption process, together with BHSiO₂ particles reduction during its' mixtures mechanical activation with betalactam substances by impact abrasive mechanization process.

The stated production process of the previously mentioned pharmaceutical composition by betalactam antibiotic powder mixture and BHSiO₂ mechanical activation with intensive impact abrasive operations allow to increase the finely divided BHSiO₂ particles (less than 5 micron) on which betalactam molecules are adsorbed and which are mostly phagocyted by macrophages [10,19].

To achieve this goal the mixture of the stated above materials in weight rating, betalactam antibiotic: BHSiO₂ equal (10-75): 1, is exposed to intensive impact abrasive mechanical activation process until the finely divided fraction weight rating is increased up to 25%.

The data from the aqueous slurry fractional makeup in terms of ceftriaxone: BHSiO₂ equal 30: 1, by the weight, measured by a laser granulometer Micro-Sizer 201is shown in picture 1 and 2.

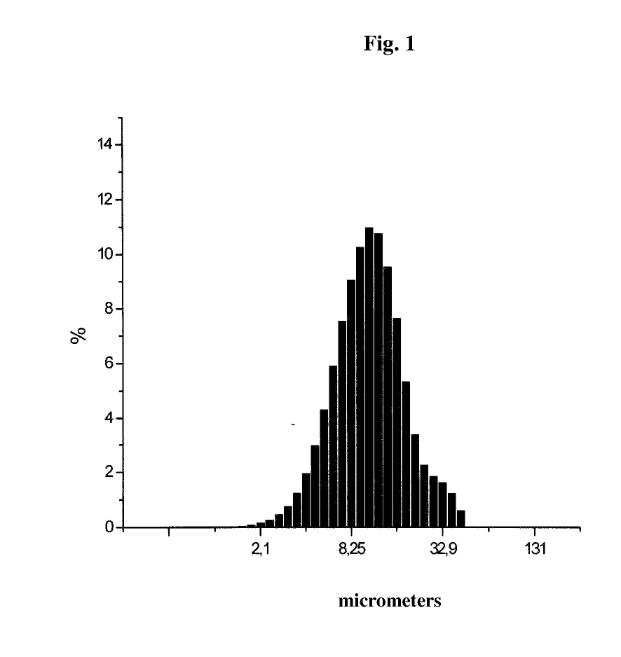

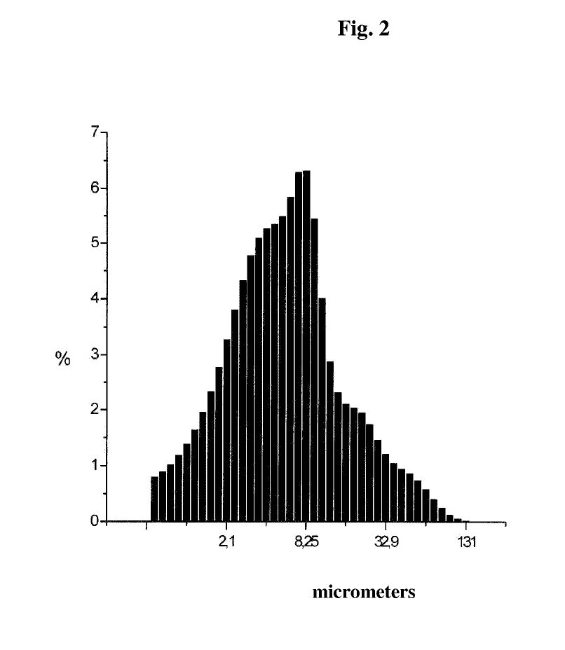

As can be seen from FIGS. 1 and 2, the two hours analyzed composition mechanical activation leads to weight rating increase of its finely dispersed fraction (particles dimension <5 micron) which contains not less than 25%.

From the received powder-like composition, one you can prepare an injection solution for parenteral insertion (water it down by any means appropriate for betalactam), composed of finely dispersed BHSiO₂ particles with inversibly sorbed any betalactan molecules on its surface.

Table [symbol] 1 contains data (received by high performance liquid chromatography method—HPLC) about different betalactam antibiotics sorption rate on BHSiO₂ particles after mechanical activation of antibiotic composition: BHSiO₂, equal 30:1, which shows that the finely dispersed nanostructured silica dioxide can be used for parenteral administration as a dosing vehicle for antibiotics and other pharmacons which are capable of sorbing on the nano- and microparticles of this inanimate matter for delivery thereof to the inflammation areas, tumor growth areas, regeneration areas, cicatrization areas, scaring areas, etc., i.e. into the areas with increased macrophages presence to purposefully increase the local concentration (as well as cellicolous) the pharmaceutical concentration and its therapeutic effect.

TABLE NO 1
Betalactam sorption rate by BHSiO₂* particles

| | Sorbed antibiotic |
|---|---|
| Composition formuation, | q-ty:BHSiO₂ q-ty, |
| m/a time** | mg (weight %) |
| Cefazolin:BHSiO₂ | 8.1 mg:16.7 mg (48%) |
| (30:1), m/a 2 hours | |
| Ceftriaxone:BHSiO₂ | 14.5 mg:16.7 mg (85%) |
| (30:1), m/a 2 hours | |
| Cefotaxime:BHSiO₂ | 9.4 mg:16.7 mg (55%) |
| (30:1), m/a 2 hours | |
| Cefuroxime:BHSiO₂ | 7.4 mg:16.7 mg (44%) |
| (30:1), m/a 2 hours | |
| Cefepime:BHSiO₂ | 16.1 mg:16.7 mg (96%) |
| (30:1), m/a 2 [symbol] | |
| Cefoperazone:BHSiO₂ | 12.2 mg:16.7 mg (73%) |
| (30:1), m/a 2 hours | |
| Cefoperazone/ | 13.9 mg:16.7 mg (83%) |
| sulbactam:BHSiO₂ | |
| (30:1), m/a 2 hours | |
| Ceftazidime:BHSiO₂ | 9.6 mg:16.7 mg (53%) |
| (30:1), m/a 2 hours | |
| Cefoxotin:BHSiO₂ | 8.5 mg:16.7 mg (51%) |
| (30:1), m/a 2 hours | |
| Meropenem:BHSiO₂ | 10.6 mg:16.7 mg (63%) |
| (30:1), m/a 2 hours | |
| Aztreonam:BHSiO₂ | 9.7 mg:16.7 mg (58%) |
| (30:1), m/a 2 hours | |
| Carbenicillin:BHSiO₂ | 11.2 mg:16.7 mg (67%) |
| (30:1), m/a 2 hours | |

*finely dispersed nanostructured silica dioxide
**mechanical activation

Introducing of the finely dispersed nanostructured silica dioxide equal betalactam: BHSiO₂ from 10:1 to 75:1 regarding its' weight is determined by the combination of 2 factors: 1) during BHSiO₂ more than 10% increase from the composition weight in case of laboratory animals, they suffer from the small capillary tube blockage of solid viscus; 2) in case of BHSiO₂ content decrease for more than 1% of the composition weight (in particular during the mice treatment of bacterial sepsis) it's therapeutic efficiency doesn't differ from the initial antibiotic basic efficiency.

To receive the composition mechanochemical method was used, which comprehends the solid components mixture processing by intensive mechanical impacts—pressure and shearing deformations, mostly realized in different kind of mills which perform impact abrasing actions on the substances. The mixture of the solid betalactam antibiotic substance and finely dispersed nanostructured silica dioxide taken in the ratio from 10:1 to 75:1 by weight, are exposed to bead mills mechanical activation. The used mixture preparation method helps in a certain way to avoid chemical degradation and achieve powder components full homogeneity in comparison with making the mixture by a simple components mixing, or evaporating their solutions, and as consequence causes a high pharmacological activity of pharmaceutical composition.

As a quantitative criterion of the minimum necessary mechanical impact dose it is comfortable to use the granulometry method of the composition suspension. It is necessary that the mass fraction of the particles less than 5 micron was more than 25%. On the other hand it is necessary to avoid the excessive mechanical processing which can cause betalactam chemical degradation which level can be controlled by the known analytical methods, such as HPLC.

Powder mixtures mechanical processing is performed in rotary, vibrational and planetary mills. As grinding bodies you can use balls, cores and etc.

Laboratory animals (mice) pharmacological tests of the compositions showed, that the mentioned compositions prepared by the mentioned method have a higher therapeutic efficiency while treating bacterial sepsis, provoked by Staphylococcus aureus, Escherichia coli and Pseudomonas aeruginosa, comparing to the initial antibiotics.

In such manner, using the mentioned pharmaceutical compositions and their production process provide the stated below advantages:
1) Clinically significant increase of the effectiveness and quality of the antimicrobial therapy of semi-acute and acute infection inflammatory diseases, death rate reduction;
2) Ecological safety, lack of wastes and low price of pharmaceutical production technology.

The proposed invention is illustrated by examples listed below.

Example [symbol] 1. Solid composition production: betalactam antibiotic—finely dispersed nanostructured silica dioxide.

The mixture of the betalactam antibiotic and BHSiO₂ in weight ratio 10:1, 20:1; 30:1 and 40:1 are being processed in an orbicular rotary mill for 1, 2 and 4 hours. The data of the water suspension granulometric composition as well as HPLC analysis of the antibiotic content (in % from the initial substance) are listed in Table [symbol] 22.

TABLE NO 2
Water suspensions granulometric composition and antibiotics
content in different composition variations

| | Dimension and content % of | | | |
|---|---|---|---|---|
| | BHSiO₂ particles** | | | Antibiotic |
| Composition content, | % < 3 | % < 5 | % < 10 | content |
| time m/a* | micron | micron | micron | (%) |
| Initial BHSiO₂ | 0.5 | 5.3 | 25.7 | — |
| Cefotaxime:BHSiO₂ | 13.4 | 30.4 | 57.3 | 89 |
| (10:1), m/a 1 hour | | | | |
| Cefotaxime:BHSiO₂ | 16.6 | 33.9 | 59.1 | 95 |
| (20:1), m/a 1 hour | | | | |
| Cefotaxime:BHSiO₂ | 13.1 | 27.7 | 47.9 | 97 |
| (40:1), m/a 1 hour | | | | |
| Cefotaxime:BHSiO₂ | 14.7 | 30.6 | 54.1 | 99 |
| (30:1), m/a 2 hours | | | | |
| Cefuroxime:BHSiO₂ | 22.6 | 35.2 | 50.2 | 97 |
| (30:1), m/a 2 hours | | | | |
| Ceftazidime:BHSiO₂ | 14.3 | 25.3 | 37.0 | 98 |
| (30:1), m/a 2 hours | | | | |
| Ceftazidime:BHSiO₂ | 23.8 | 38.9 | 56.2 | 96 |
| (30:1), m/a 4 hours | | | | |
| Cefepime:BHSiO₂ | 23.8 | 38.8 | 57.7 | 92 |
| (30:1), m/a 2 hours | | | | |
| Ceftriaxone:BHSiO₂ | 24.2 | 43.9 | 66.2 | 97 |
| (30:1), m/a 1 hour | | | | |
| Ceftriaxone:BHSiO₂ | 19.4 | 34.5 | 52.4 | 99 |
| (30:1), m/a 2 hours | | | | |
| Ceftriaxone:BHSiO₂ | 14.5 | 26.4 | 41.7 | 95 |
| (30:1), m/a 4 hours | | | | |
| Ceftriaxone:BHSiO₂ | 23.4 | 41.2 | 59.1 | 98 |
| (40:1), m/a 1 hour | | | | |
| Aztreonam:BHSiO₂ | 21.7 | 39.4 | 53.6 | 97 |
| (30:1), m/a 2 hours | | | | |
| Meropenem:BHSiO₂ | 19.1 | 32.9 | 47.3 | 98 |
| (30:1), m/a 2 hours | | | | |
| Aztreonam:BHSiO₂ | 19.8 | 31.1 | 49.5 | 97 |
| (30:1), m/a 2 hours | | | | |
| Carbenicillin:BHSiO₂ | 22.3 | 38.9 | 51.4 | 96 |
| (30:1), m/a 2 hours | | | | |

*finely dispersed nanostructured silica dioxide
**mechanical activation

As can be seen from table [symbol] 2, the chosen conditions of the composition production afford to increase until a certain value (not less than 25% from the total weight) the part of the finely dispersed BHSiO₂ fraction (particles size less than 5 micron) and to avoid the antibiotic chemical degradation.

Example [symbol] 2. Determination of the therapeutic efficiency of antimicrobial preparations and pharmaceutical compositions.

There has been a research of betalactam antibiotics (Cefazolin, Cefuroxime, Cefotaxime, Ceftriaxone, Cefoperazone, Cefoperazone/sulbactam, Ceftazidime, Cefepime, Cefoxitin, Aztreonam, Meropenem, Carbenicillin) and their compositions mechanized for 2 hours and composed of a mixture antibiotic/BHSiO₂ in weight ratio 30:1, consequently (Cefazolin/BHSiO₂, Cefuroxime/BHSiO₂, Cefotaxime/BHSiO₂, Ceftriaxone/BHSiO₂, Cefoperazone/BHSiO₂, Cefoperazone/sulbactam/BHSiO₂, Ceftazidime/BHSiO₂, Cefepime/BHSiO₂, Cefoxitin/BHSiO₂, Aztreonam/BHSiO₂, Meropenem/BHSiO₂, Carbenicillin/BHSiO₂).

To determine therapeutic efficiency of betalactam and their pharmaceutical compositions including BHSiO₂, we used experimental sepsis models and a statistical processing method of the received data (χ²) according to [22, 23].

Microorganisms: Staphylococcus aureus (ATCC [symbol] 25923 F-49), Escherichia coli (ATCC [symbol] 25922 F-50), Pseudomonas aeruginosa (ATCC [symbol] 27853 F-51).

Animals: for the experiments we used hybrid mice (CBA×C₅₇Black/₆)CBF₁ according to the “Regulations for test animals use” (USSR Ministry of health order supplement [symbol] 755 from 12.08. 1977).

Experimental sepsis models:

The mice have been injected 0.8 ml of Pseudomonas aeruginosa daily culture suspension with a dosage 5×10⁸ CFU/mouse or Staphylococcus aureus daily culture suspension with a dosage of 10¹⁰ CFU/mouse or Escherichia coli daily culture suspension with a dosage of 8×10⁸ CFU/mouse.

The control group has been injected with 0.8 ml of normal saline solution (0.9% sodium chloride solution). In a day after being infected the test mice have been daily (during 3 days) intravenous injected with 100 mg/kg of antibiotics or different pharmaceutical compositions (antibiotic/BHSiO₂) watered down with 0.25 ml of normal saline solution. The control group of mice has been injected using the same scheme with normal saline solution 0.25 mg.

Antibacterial therapy efficiency was evaluated basing on the quantity of the surviving animals on the 7th day after being infected [22, 23].

The obtained data shown in Table [symbol] 3 reflect the results of 3 independent experiments (for each preparation research were used not less than 30 test animals in total).

TABLE NO 3
Bacterial sepsis antimicrobial therapy efficiency

| | Mice survival rate on the 7^{th} day of infection** | | | |
|---|---|---|---|---|
| Tested antibiotics and | Staphylococcus | Escherichia | Pseudomonas | |
| compositions* | aureus | coli | aeruginosa | χ₂ |
| Normal saline solution | 0% (0/30) | 0% (0/30) | 0% (0/30) | — |
| (control) | | | | |
| Cefazolin | 37.5% (12/32) | — | —*** | P < 0.01 |
| Cefazolin/BHSiO₂ | 83.9% (26/31) | — | — | |
| Cefuroxime | 40.0% (14/35) | 43.7% (14/32) | — | P < 0.01 |
| Cefuroxime/BHSiO₂ | 84.4% (27/32) | 81.2% (26/32) | — | |
| Cefotaxime | 40.0% (12/30) | 43.3% (13/30) | — | P < 0.01 |
| Cefotaxime/BHSiO₂ | 86.7% (26/30) | 83.3% (25/30) | — | |
| Ceftriaxone | 46.7% (14/30) | 41.9% (13/31) | — | P < 0.01 |
| Ceftriaxone/BHSiO₂ | 90.0% (27/30) | 87.5% (28/32) | — | |
| Cefoperazone | — | 45.2% (14/31) | 40.0% (12/30) | P < 0.01 |
| Cefoperazone/BHSiO₂ | — | 90.0% (27/30) | 80.6% (25/31) | |
| Ceftazidime | — | 38.7% (15/31) | 43.3% (13/30) | P < 0.01 |
| Ceftazidime/BHSiO₂ | — | 84.8% (28/33) | 86.7% (26/30) | |
| Cefepime | 46.7% (14/30) | 43.7% (14/32) | 46.7% (14/30) | P < 0.01 |
| Cefepime/BHSiO₂ | 90.0% (27/30) | 85.3% (29/34) | 90.3% (28/31) | |
| Cefoxitin | 35.2% (15/34) | 46.7% (14/30) | — | P < 0.01 |
| Cefoxitin/BHSiO₂ | 87.5% (28/32) | 83.3% (25/30) | — | |
| Aztreonam | — | 77.5% (31/40) | 74.4% (32/43) | P < 0.01 |
| Aztreonam/BHSiO₂ | — | 95.0% (38/40) | 95.2% (40/42) | |
| Meropenem | 73.3% (22/30) | 78.0% (32/41) | 73.8% (31/42) | P < 0.01 |
| Meropenem/BHSiO₂ | 90.6% (29/32) | 95.0% (38/40) | 95.1% (39/41) | |
| Carbenicillin | 46.7% (14/30) | 43.3% (13/30) | 43.3% (13/30) | P < 0.01 |
| Carbenicillin/BHSiO₂ | 83.3% (25/30) | 86.7% (26/30) | 90.0% (27/30) | |
| Cefoperazone/sulbactam | 56.7% (17/30) | 58.1% (18/31) | 59.3% (19/32) | P < 0.01 |
| Cefoperazone/sulbactam | 86.7% (26/30) | 93.3% (28/30) | 93.5% (29/31) | |
| BHSiO₂ | | | | |

*mixtures composed of betalactam antibiotic:finely dispersed nanostructured silica dioxide (BHSiO₂) in weight ratio 30:1
**survival rate/infected animals rate measured in % and absolute values
***tests were not conducted because microorganisms relatively low-grade sensitivity to initial antibiotics

As may be seen from Table [symbol] 3 all suggested antimicrobial action pharmaceutical compositions (betalactsm/BHSiO₂) definitely possess an increased therapeutic efficiency (1,2-2 times higher) comparing to simple betalactam in case of lab animals sepsis treatment, provoked by Pseudomonas aeruginosa, Staphylococcus aureus or Escherichia coli. These results mostly concern compositions with cefalosporins, cefamicyns and penicillins used as betalactam.

### USED LITERATURE

1. Antibacterial pharmacons. Preparations standartization methods.—M.: JSC [symbol] Medicine [symbol] Publishing [symbol] , 2004.—944 p.
2. M. D. Mashkovsky//Pharmacons: Tome 2.-14^{th} edition. M.: LLC [symbol] Novaya Volna Publishing [symbol] , 2001.—608 p.
3. Patent RU [symbol] 2377985 MΠK A61K31/43
4. Rational antibacterial pharmacopy//Practicians Guidance. Under the general editorship of V. P. Yakovlev, S. V. Yakovlev.—M.: Litterra, 2003.—1008 p.
5. A. M. Mayansky//Microbiology for physitians (patogenetic microbiology essays).—Nizhny Novgorod: Nizhny Novgorod State Medical Academy Publishing, 1999.—400 p.
6. Abeylath S. C., Turos E. Drug delivery approaches to overcome bacterial resistance to β-lactam antibiotics//Expert Opinion on Drug Delivery.—2008.—Vol. 5.—P. 931-949.
7. Bastus N. G., Sanchez-Tillo E., Pujals S. et al. Peptides conjugated to gold nanopar-ticles induce macrophage activation//Molecular Immunology.—2009.—Vol. 46.—P. 743-748.
8. Pinto-Alphandary H., Andremont A., Couvreur P. Targeted delivery of antibiotics using liposomes and nanoparticles: research and applications//International Journal of Antimicrobial Agents.—2000.—Vol. 13.—P. 155-168.
9. Ulbrich W., Lamprech A. Targeted drug-delivery approaches by nanoparticulate carriers in the therapy of inflammatory diseases//Journal Royal Society Interface.—2010.—Vol. 7, Suppl. 1.—P. S55-S66.
10. A. E. Guliaev, B. A. Ermekbaeva, G. Y. Kivman and etc. Nanoparticles as targeted antibiotic transport (review)//Chemical and pharmaceutical magazine.—1998.— [symbol] 3.—P. 3-6.
11. Rosemary M. J., MacLaren I., Pradeep T. Investigation of antibacterial properties of ciprofloxacin@SiO₂.//Langmuir.—2006.—Vol. 22.—P. 10125-10129.
12. Rai A., Prabhune A., Perry C. C. Antibiotic mediated synthesis of gold nanoparticles with potent antimicrobial activity and their application in antimicrobial coatings//Journal of Materials Chemistry.—2010.—Vol. 20.—P. 6789-6798.

13. Park J-H., Gu L., Maltzahn G. et al. Biodegradable luminescent porous silica nanoparticles for in vivo applications//Nature Materials.—2009.—Vol. 8.—P. 331-336.
14. Pernis B. Silica and the immune system//Acta Biomed.—2005.—Vol. 76, Suppl. 2.—P. 38-44.
15. Tasciotti E., Liu X., Bhavane R. Et et al. Mesoporous silica particles as a multistage delivery system for imaging and therapeutic applications//Nature Nanotechnology.—2008.—Vol. 3.—P. 151-157.
16. Seleem M. N., Munusamy P., Ranjan A et al. Silica-antibiotic hybrid nanoparticles for targeting intracellular pathogens//Antimicrobial Agents and Chemotherapy.—2009.—Vol. 53.—P. 4270-4274.
17. Clinical chemistry and silica dioxide clinical use//Edited by NAS of Ukrane academician F. F. Chuyko—Kiev: [symbol] Naukova Dumka [symbol] , 2003.—416 p.
18. Chuiko A., Pentyuk A., Shtat'ko E., Chuiko N. Medical aspects of application of highly disperse amorphous silica//Surface Chemistry in Biomedical and Environmental Science. Edited by J. P. Blitz and V. Gun'ko.II. Mathematics, Physics and Chemistry.—2006.—Vol. 228.—P. 191-204.
19. Lucarelli M., Gatti A. M., Savarino G. et al. Innate defence functions of macrophages can be biased by nano-sized ceramic and metallic particles//European Cytokine Network.—2004.—Vol. 15.—P. 339-346.
20. Zolnik B. S., Gonzalez-Fernandez A., Sadrieh N., Dobrovolskaia V. Minireview: Nanoparticles and the immune system//Endocrinology.—2010.—Vol. 151.—P. 458-465.
21. N. A. Piataev, F. N. Beliaev, M. D. Romanov, I. S. Kotlov//Pharmacons directed celll assosiated transport.—Saransk: Mordovia University Publishing, 2007.—140 p.
22. Eckhardt C., Fickweiler K., Schaumann R. et al. Therapeutic efficacy of moxifloxacin in a murine model of severe systemic mixed infection with E. coli and B. fragilis//Anaerobe.—2003.—Vol. 9.—P. 157-160.
23. Schaumann R., Blatz R., Beer J. et al. Effect of moxifloxacin versus imipenem/cilastatin treatment on the mortality of mice infected intravenously with different strains of Bacteroides fragilis and Escherichia coli//Journal of Antimicrobial Chemotherapy.—2004.—Vol. 53.—P. 318-324.

1. An antimicrobial pharmaceutical composition for parenteral administration made in the form of injection preparation powder, said composition including: a first amount of betalactam antibiotic acting as a therapeutic agent, and a second amount of finely dispersed nanostructured silica dioxide; wherein said composition is characterized by a weight ratio of the first amount to the second amount, and said weight ratio constituting: (10-75): 1.

2. The composition according to claim 1, wherein said finely dispersed nanostructured silica dioxide is composed of particles; said finely dispersed nanostructured silica dioxide includes a portion of at least 25% thereof, wherein said portion substantially consists of said particles having a size not exceeding 5 micrometers.

3. A process of production of an antimicrobial pharmaceutical composition for parenteral administration comprising the steps of:
providing a first amount of betalactam antibiotic in the form of powder;
providing a second amount of finely dispersed nanostructured silica dioxide in the form of powder;
mixing said first amount with said second amount in a weight ratio of (10-75): 1, thereby obtaining a mixture; and
subjecting said mixture to mechanical processing by means of blow-abrasive actions.

4. The process according to claim 3, wherein said finely dispersed nanostructured silica dioxide is composed of particles; said blow-abrasive actions are resulted in that said finely dispersed nanostructured silica dioxide includes a portion of at least 25% thereof, wherein said portion substantially consists of said particles having a size not exceeding 5 micrometers.
